(19) United States
(12) Patent Application Publication
(10) Pub. No.: US 2025/0210182 A1
(43) Pub. Date: Jun. 26, 2025

(54) INFORMATION PROCESSING APPARATUS, INFORMATION PROCESSING METHOD, AND INFORMATION PROCESSING PROGRAM

(71) Applicant: FUJIFILM CORPORATION, Tokyo (JP)

(72) Inventors: Keigo NAKAMURA, Tokyo (JP); Yohei MOMOKI, Tokyo (JP)

(21) Appl. No.: 19/079,471

(22) Filed: Mar. 14, 2025

Related U.S. Application Data
(63) Continuation of application No. PCT/JP2023/035274, filed on Sep. 27, 2023.

(30) Foreign Application Priority Data
Sep. 27, 2022 (JP) 2022-154170
Sep. 22, 2023 (JP) 2023-159178

(51) Int. Cl. G16H 30/40; G06T 7/00; G06V 10/25; G16H 30/20; G16H 50/20; G16H 50/70

(52) U.S. Cl. CPC G16H 30/40; G06T 7/0012; G06V 10/25; G16H 30/20; G16H 50/20; G16H 50/70; G06T 2207/30096

(57) ABSTRACT

An information processing apparatus including a processor, wherein the processor is configured to: acquire an image; generate at least one piece of image finding information, which is information indicating findings in the image, based on the image; and search for at least one comment on findings related to the image finding information from a database in which a plurality of comments on findings are registered in advance.

### CROSS REFERENCE TO RELATED APPLICATIONS

This application is a continuation of International Application No. PCT/JP2023/035274, filed on Sep. 27, 2023, which claims priority from Japanese Patent Application No. 2022-154170, filed on Sep. 27, 2022, and No. 2023-159178, filed on Sep. 22, 2023. The entire disclosure of each of the above applications is incorporated herein by reference.

### BACKGROUND

### Technical Field

The present disclosure relates to an information processing apparatus, an information processing method, and an information processing program.

### Related Art

In the related art, image diagnosis is performed using medical images obtained by imaging apparatuses such as computed tomography (CT) apparatuses and magnetic resonance imaging (MRI) apparatuses. Further, medical images are analyzed via computer aided detection/diagnosis (CAD) using a discriminator in which learning is performed by deep learning or the like, and regions of interest including structures, lesions, and the like included in the medical images are detected and/or diagnosed. The medical images and analysis results via CAD are transmitted to a terminal of a healthcare professional such as a radiologist who interprets the medical images. The healthcare professional such as a radiologist interprets the medical images with reference to the medical images and analysis results using his or her own terminal and creates an interpretation report.

In addition, various methods for supporting the creation of interpretation reports have been proposed. For example, JP2019-153250A discloses a technology for creating an interpretation report based on a keyword input by a radiologist and on an analysis result of a medical image. In the technology disclosed in JP2019-153250A, a sentence to be included in the interpretation report is created by using a recurrent neural network trained to generate a sentence from input characters.

Furthermore, for example, JP2008-052544A discloses a technology for searching a database for similar images and related reports based on search standard image data. In the technology disclosed in JP2008-052544A, character information constituting an interpretation report on image data is added as metadata for search and stored in a database. At the time of a search, as the radiologist designates search standard image data, the search is executed using character information already added to the search standard image data as a keyword.

The method of expressing a sentence described in an interpretation report may differ depending on the creator of the interpretation report (for example, the radiologist). For example, one radiologist may express an object as a “20 mm nodule”, whereas another radiologist may express it as a “nodule (20 mm)”. With support technologies for creating interpretation reports in the related art, even in a case in which there was no problem with the content of the sentence generated by a computer, it was sometimes necessary to take the time and effort to correct the expression. Therefore, there is a demand for a technology that can support the creation of an interpretation report based on the image to be interpreted, without causing the time and effort to correct the method of expressing the sentence.

### SUMMARY

The present disclosure provides an information processing apparatus, an information processing method, and an information processing program that can support creation of an interpretation report.

According to a first aspect of the present disclosure, there is provided an information processing apparatus comprising a processor, in which the processor is configured to: acquire an image; generate at least one piece of image finding information, which is information indicating findings in the image, based on the image; and search for at least one comment on findings related to the image finding information from a database in which a plurality of comments on findings are registered in advance.

According to a second aspect of the present disclosure, in the first aspect, the processor may be configured to: receive an input of at least one piece of narrowed-down finding information related to the image; and generate, as the image finding information, information indicating findings of the image related to the narrowed-down finding information.

According to a third aspect of the present disclosure, in the second aspect, the processor may be configured to search the database for a comment on findings related to at least one of the image finding information or the narrowed-down finding information.

According to a fourth aspect of the present disclosure, in the second or third aspect, the processor may be configured to: receive an input of a partial comment on findings which is a part of a comment on findings related to the image and includes the narrowed-down finding information; and specify the narrowed-down finding information included in the partial comment on findings.

According to a fifth aspect of the present disclosure, in any one of the first to fourth aspects, the processor may be configured to correct the comment on findings obtained through the search based on the image.

According to a sixth aspect of the present disclosure, in any one of the first to fifth aspects, the processor may be configured to: acquire at least one of a past image obtained by capturing a subject of the image in the past or at least one piece of past finding information which is information indicating findings in the past image; and generate the image finding information indicating changes in findings over time based on the image and at least one of the past image or the past finding information.

According to a seventh aspect of the present disclosure, in any one of the first to sixth aspects, the processor may be configured to: extract at least one region of interest included in the image; and generate information indicating findings of the extracted region of interest as the image finding information.

According to an eighth aspect of the present disclosure, in the seventh aspect, the processor may be configured to, in a case in which a plurality of regions of interest included in the image are extracted: generate the image finding information for each region of interest; and search the database for comments on findings related to a plurality of pieces of the image finding information for each region of interest.

According to a ninth aspect of the present disclosure, in any one of the first to sixth aspects, the processor may be configured to: receive a designation of at least one region of interest included in the image; and generate information indicating findings of the designated region of interest as the image finding information.

According to a tenth aspect of the present disclosure, in the ninth aspect, the processor may be configured to, in a case in which a designation of a plurality of regions of interest included in the image is received: generate the image finding information for each region of interest; and search the database for comments on findings related to a plurality of pieces of the image finding information for each region of interest.

According to an eleventh aspect of the present disclosure, in any one of the first to tenth aspects, the processor may be configured to: specify, for each of the plurality of comments on findings registered in the database, finding information related to a comment on findings and add the finding information to the comment on findings; and search the database for a comment on findings to which finding information that is the same as or similar to the image finding information has been added.

According to a twelfth aspect of the present disclosure, in the eleventh aspect, the processor may be configured to, in a case in which each of two or more comments on findings registered in the database includes a synonym, add the same finding information to each of the two or more comments on findings.

According to a thirteenth aspect of the present disclosure, in any one of the first to twelfth aspects, the plurality of comments on findings and key images related to the comments on findings may be registered in advance in the database in association with each other, and the processor may be configured to display, on a display, the comment on findings obtained through the search and the key image related to the comment on findings in association with each other.

According to a fourteenth aspect of the present disclosure, in any one of the first to thirteenth aspects, the processor may be configured to: calculate a rate of match between the image finding information and finding information included in the searched-for comment on findings; and determine a display method for the searched-for comment on findings based on the rate of match.

According to a fifteenth aspect of the present disclosure, in any one of the first to fourteenth aspects, the processor may be configured to, in a case in which a comment on findings for a past image obtained by capturing a subject of the image in the past is registered in the database, preferentially search for the comment on findings.

According to a sixteenth aspect of the present disclosure, in any one of the first to fifteenth aspects, creator information indicating a creator of a comment on findings may be added to the plurality of comments on findings registered in the database, and the processor may be configured to: receive a designation of a creator of a comment on findings to be searched; and in a case in which a comment on findings to which the creator information indicating the designated creator is added is registered in the database, preferentially search for the comment on findings.

According to a seventeenth aspect of the present disclosure, in any one of the first to sixteenth aspects, the image may be a medical image, and the image finding information may indicate at least one of a type, a property, a position, a measurement value, or an estimated disease name of a lesion included in the medical image.

According to an eighteenth aspect of the present disclosure, in any one of the second to fourth aspects, the image may be a medical image, and the narrowed-down finding information may indicate an estimated disease name of a lesion included in the medical image.

According to a nineteenth aspect of the present disclosure, there is provided an information processing method executed by a computer, the information processing method comprising: acquiring an image; generating at least one piece of image finding information, which is information indicating findings in the image, based on the image; and searching for at least one comment on findings related to the image finding information from a database in which a plurality of comments on findings are registered in advance.

According to a twentieth aspect of the present disclosure, there is provided an information processing program for causing a computer to execute: acquiring an image; generating at least one piece of image finding information, which is information indicating findings in the image, based on the image; and searching for at least one comment on findings related to the image finding information from a database in which a plurality of comments on findings are registered in advance.

The information processing apparatus, the information processing method, and the information processing program according to the aspects of the present disclosure can support creation of an interpretation report.

### BRIEF DESCRIPTION OF THE DRAWINGS

FIG. 1 is a diagram showing an example of a schematic configuration of an information processing system.

FIG. 2 is a diagram showing an example of a medical image.

FIG. 3 is a diagram showing an example of a medical image.

FIG. 4 is a block diagram showing an example of a hardware configuration of an information processing apparatus.

FIG. 5 is a block diagram showing an example of a functional configuration of the information processing apparatus.

FIG. 6 is a diagram showing an example of information registered in a report DB.

FIG. 7 is a diagram showing an example of a dictionary.

FIG. 8 is a diagram showing an example of a screen displayed on a display.

FIG. 9 is a diagram showing an example of a screen displayed on a display.

FIG. 10 is a flowchart showing an example of first information processing.

FIG. 11 is a diagram showing an example of a screen displayed on a display.

FIG. 12 is a diagram showing an example of a table.

FIG. 13 is a diagram showing an example of a screen displayed on a display.

FIG. 14 is a flowchart showing an example of second information processing.

### DETAILED DESCRIPTION

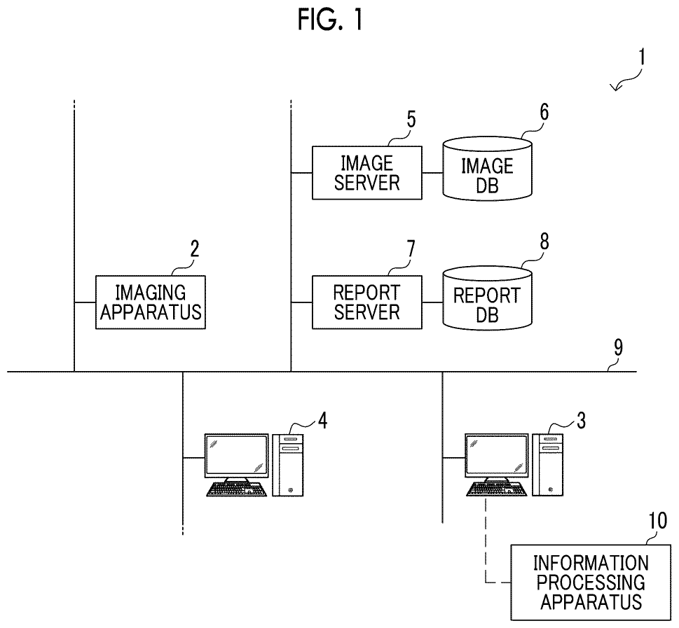

Hereinafter, an exemplary embodiment of the present disclosure will be described with reference to the drawings. First, a configuration of an information processing system 1 to which an information processing apparatus 10 according to an aspect of the present disclosure is applied will be described. FIG. 1 is a diagram showing a schematic configuration of the information processing system 1. The information processing system 1 shown in FIG. 1 performs imaging of an examination target part of a subject and storing of a medical image acquired by the imaging based on an examination order from a doctor in a medical department using a known ordering system. In addition, the information processing system 1 performs interpretation work of a medical image and creation of an interpretation report by a radiologist and viewing of the interpretation report by a doctor of a medical department that is a request source.

As shown in FIG. 1, the information processing system 1 includes an imaging apparatus 2, an interpretation workstation (WS) 3 that is an interpretation terminal, a medical care WS 4, an image server 5, an image database (DB) 6, a report server 7, and a report DB 8. The imaging apparatus 2, the interpretation WS 3, the medical care WS 4, the image server 5, the image DB 6, the report server 7, and the report DB 8 are connected to each other via a wired or wireless network 9 in a communicable state.

Each apparatus is a computer on which an application program for causing each apparatus to function as a component of the information processing system 1 is installed. The application program may be recorded on, for example, a recording medium, such as a digital versatile disc read only memory (DVD-ROM) or a compact disc read only memory (CD-ROM), and distributed, and be installed on the computer from the recording medium. In addition, the application program may be stored in, for example, a storage device of a server computer connected to the network 9 or in a network storage in a state in which it can be accessed from the outside, and be downloaded and installed on the computer in response to a request.

The imaging apparatus 2 is an apparatus (modality) that generates a medical image T showing a diagnosis target part of the subject by imaging the diagnosis target part. Examples of the imaging apparatus 2 include a simple X-ray imaging apparatus, a computed tomography (CT) apparatus, a magnetic resonance imaging (MRI) apparatus, a positron emission tomography (PET) apparatus, an ultrasound diagnostic apparatus, an endoscope, a fundus camera, and the like. The medical image generated by the imaging apparatus 2 is transmitted to the image server 5 and is stored in the image DB 6.

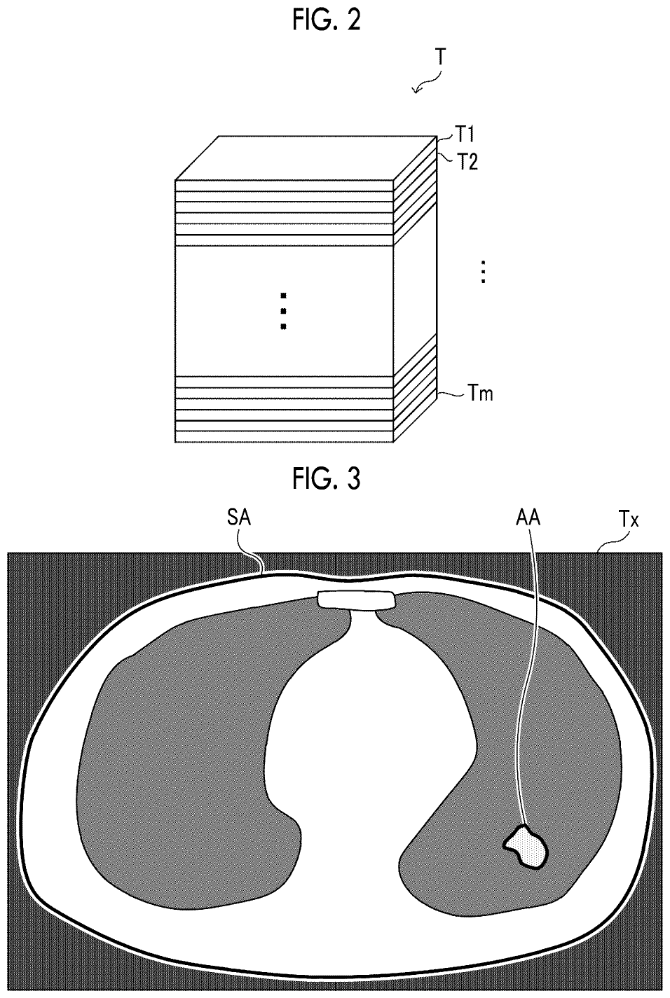

FIG. 2 is a diagram schematically showing an example of a medical image acquired by the imaging apparatus 2. A medical image T shown in FIG. 2 is, for example, a CT image consisting of a plurality of tomographic images T1 to Tm (m is 2 or more) representing tomographic planes from a head to a waist of one subject (human body). The medical image T is an example of an image of the present disclosure.

FIG. 3 is a diagram schematically showing an example of one tomographic image Tx out of the plurality of tomographic images T1 to Tm. The tomographic image Tx shown in FIG. 3 represents a tomographic plane including lungs. Each of the tomographic images T1 to Tm may include a region SA of a structure showing various organs and viscera of the human body (for example, lungs, livers, and the like), various tissues constituting various organs and viscera (for example, blood vessels, nerves, muscles, and the like), and the like. In addition, each tomographic image may include a lesion region AA such as, for example, nodules, tumors, injuries, defects, and inflammation. In the tomographic image Tx shown in FIG. 3, a lung region is the region SA of the structure, and a nodule region is the lesion region AA. A single tomographic image may include regions SA of a plurality of structures and/or lesion regions AA. Hereinafter, at least one of the region SA of the structure included in the medical image or the lesion region AA included in the medical image will be referred to as a “region of interest”.

The interpretation WS 3 is a computer used by, for example, a healthcare professional such as a radiologist of a radiology department to interpret a medical image and to create an interpretation report, and encompasses an information processing apparatus 10 according to the present exemplary embodiment. In the interpretation WS 3, a viewing request for a medical image to the image server 5, various types of image processing for the medical image received from the image server 5, display of the medical image, and input reception of a sentence regarding the medical image are performed. In the interpretation WS 3, analysis processing for medical images, support in creating an interpretation report based on the analysis result, a registration request and a viewing request for the interpretation report to the report server 7, and display of the interpretation report received from the report server 7 are performed. The above processes are performed by the interpretation WS 3 executing software programs for respective processes.

The medical care WS 4 is a computer used by, for example, a healthcare professional such as a doctor in a medical department to observe a medical image in detail, view an interpretation report, create an electronic medical record, and the like, and is configured to include a processing device, a display device such as a display, and input devices such as a keyboard and a mouse. In the medical care WS 4, a viewing request for the medical image to the image server 5, display of the medical image received from the image server 5, a viewing request for the interpretation report to the report server 7, and display of the interpretation report received from the report server 7 are performed. The above processes are performed by the medical care WS 4 executing software programs for respective processes.

The image server 5 is a general-purpose computer on which a software program that provides a function of a database management system (DBMS) is installed. The image server 5 is connected to the image DB 6. The connection form between the image server 5 and the image DB 6 is not particularly limited, and may be a form connected by a data bus, or a form connected to each other via a network such as a network attached storage (NAS) and a storage area network (SAN).

The image DB 6 is realized by, for example, a storage medium such as a hard disk drive (HDD), a solid state drive (SSD), and a flash memory. In the image DB 6, the medical image acquired by the imaging apparatus 2 and accessory information attached to the medical image are registered in association with each other.

The accessory information may include, for example, identification information such as an image identification (ID) for identifying a medical image, a tomographic ID assigned to each tomographic image included in the medical image, a subject ID for identifying a subject, and an examination ID for identifying an examination. In addition, the accessory information may include, for example, information related to imaging such as an imaging method, an imaging condition, an imaging purpose, an imaging location, and an imaging date and time related to imaging of a medical image. The “imaging method” and “imaging condition” are, for example, a type of the imaging apparatus 2, an imaging part, an imaging protocol, an imaging sequence, an imaging method, the presence or absence of use of a contrast medium, a slice thickness in tomographic imaging, and the like. In addition, the accessory information may include information related to the subject such as the name, date of birth, age, and gender of the subject.

In a case in which the image server 5 receives a request to register a medical image from the imaging apparatus 2, the image server 5 prepares the medical image in a format for a database and registers the medical image in the image DB 6. In addition, in a case in which the viewing request from the interpretation WS 3 and the medical care WS 4 is received, the image server 5 searches for a medical image registered in the image DB 6 and transmits the searched-for medical image to the interpretation WS 3 and to the medical care WS 4 that are viewing request sources.

The report server 7 is a general-purpose computer on which a software program that provides a function of a database management system is installed. The report server 7 is connected to the report DB 8. The connection form between the report server 7 and the report DB 8 is not particularly limited, and may be a form connected by a data bus or a form connected via a network such as a NAS and a SAN.

The report DB 8 is realized by, for example, a storage medium such as an HDD, an SSD, and a flash memory. In the report DB 8, an interpretation report created in the interpretation WS 3 is registered (details will be described later). The report DB 8 is an example of a database of the present disclosure.

Further, in a case in which the report server 7 receives a request to register the interpretation report from the interpretation WS 3, the report server 7 prepares the interpretation report in a format for a database and registers the interpretation report in the report DB 8. Further, in a case in which the report server 7 receives the viewing request for the interpretation report from the interpretation WS 3 and the medical care WS 4, the report server 7 searches for the interpretation report registered in the report DB 8, and transmits the searched-for interpretation report to the interpretation WS 3 and to the medical care WS 4 that are viewing request sources.

The network 9 is, for example, a network such as a local area network (LAN) and a wide area network (WAN). The imaging apparatus 2, the interpretation WS 3, the medical care WS 4, the image server 5, the image DB 6, the report server 7, and the report DB 8 included in the information processing system 1 may be disposed in the same medical institution, or may be disposed in different medical institutions or the like. Further, the number of each apparatus of the imaging apparatus 2, the interpretation WS 3, the medical care WS 4, the image server 5, the image DB 6, the report server 7, and the report DB 8 is not limited to the number shown in FIG. 1, and each apparatus may be composed of a plurality of apparatuses having the same functions.

The method of expressing a sentence described in an interpretation report may differ depending on the creator of the interpretation report (for example, the radiologist). For example, one radiologist may express an object as a “20 mm nodule”, whereas another radiologist may express it as a “nodule (20 mm)”. Therefore, the information processing apparatus 10 according to the present exemplary embodiment supports the creation of an interpretation report based on the image to be interpreted, without causing the time and effort to correct such sentence expressions. The information processing apparatus 10 will be described below. As described above, the information processing apparatus 10 is encompassed in the interpretation WS 3.

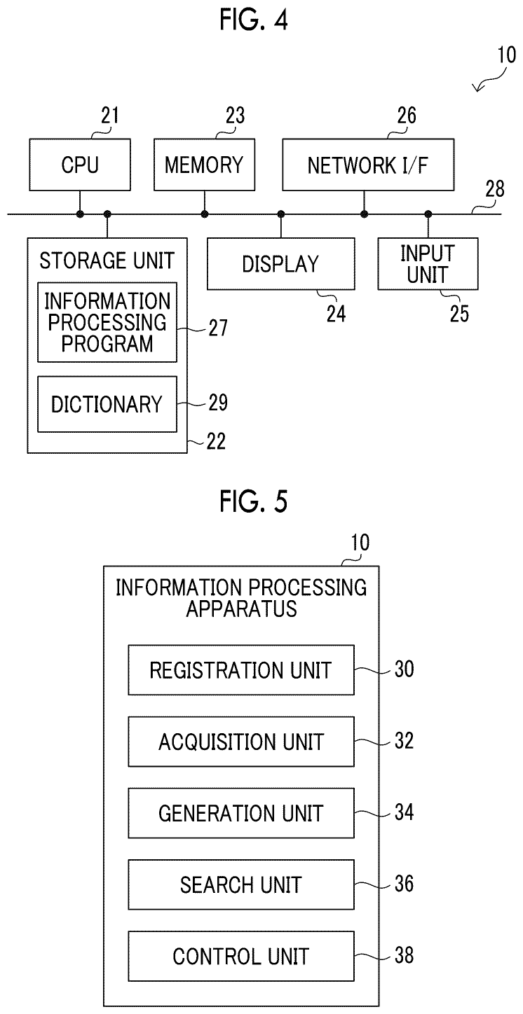

First, with reference to FIG. 4, an example of a hardware configuration of the information processing apparatus 10 according to the present exemplary embodiment will be described. As shown in FIG. 4, the information processing apparatus 10 includes a central processing unit (CPU) 21, a non-volatile storage unit 22, and a memory 23 as a temporary storage area. Further, the information processing apparatus 10 includes a display 24 such as a liquid-crystal display, an input unit 25 such as a keyboard and a mouse, and a network interface (I/F) 26. The network I/F 26 is connected to the network 9 and performs wired and/or wireless communication. The CPU 21, the storage unit 22, the memory 23, the display 24, the input unit 25, and the network I/F 26 are connected to each other via a bus 28 such as a system bus and a control bus so that various types of information can be exchanged.

The storage unit 22 is realized by, for example, a storage medium such as an HDD, an SSD, and a flash memory. An information processing program 27 in the information processing apparatus 10 is stored in the storage unit 22. The CPU 21 reads out the information processing program 27 from the storage unit 22, loads the read-out program into the memory 23, and executes the loaded information processing program 27. The CPU 21 is an example of a processor of the present disclosure. Also, the storage unit 22 stores a dictionary 29 (details will be described later). As the information processing apparatus 10, for example, a personal computer, a server computer, a smartphone, a tablet terminal, a wearable terminal, or the like can be applied as appropriate.

Next, with reference to FIGS. 5 to 9, an example of a functional configuration of the information processing apparatus 10 according to the present exemplary embodiment will be described. As shown in FIG. 5, the information processing apparatus 10 includes a registration unit 30, an acquisition unit 32, a generation unit 34, a search unit 36, and a control unit 38. In a case in which the CPU 21 executes the information processing program 27, the CPU 21 functions as each of the functional units, namely, the registration unit 30, the acquisition unit 32, the generation unit 34, the search unit 36, and the control unit 38.

### (Pre-Processing)

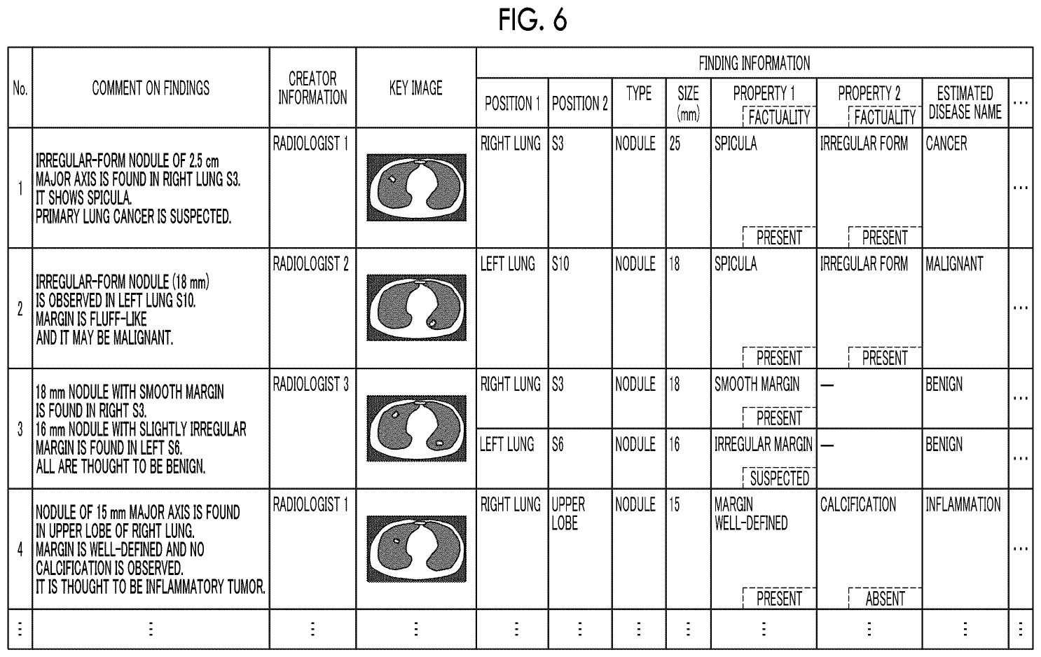

First, pre-processing in the report DB 8 that is executed by the information processing apparatus 10 prior to supporting the creation of an interpretation report will be described. As described above, in the report DB 8, created interpretation reports are registered. FIG. 6 shows an example of information included in an interpretation report registered in the report DB 8. As shown in FIG. 6, in the report DB 8, a plurality of comments on findings, creator information indicating the creator of the comment on findings, a key image related to the comment on findings, and finding information related to the comment on findings are registered in association with each other.

The comments on findings, the creator information, and the key image are registered in the report DB 8 by the report server 7 when the interpretation report is created. For case of understanding, FIG. 6 shows the key image registered in the report DB 8, but the registration destination of the key image may be another database such as the image DB 6. In this case, an address indicating the registration destination of the key image may be registered in the report DB 8 instead of the key image itself.

The registration unit 30 specifies, each of a plurality of comments on findings registered in the report DB 8, finding information related to the comments on findings and adds the specified finding information to the comments on findings. FIG. 6 shows a state in which the finding information is added to each comment on findings. For example, the finding information is information indicating at least one of various findings such as a type (name), a property, a position, a measurement value, or an estimated disease name of a region of interest included in the medical image.

Examples of types (names) include the names of structures such as “lung” and “liver”, and the names of lesions such as “nodule”. The property mainly means the features of the lesion. For example, in the case of a lung nodule, findings indicating opacity such as “solid” and “ground-glass”, margin shapes such as “well-defined/ill-defined”, “smooth/irregular”, “spicula”, “lobulated”, and “lagged”, and an overall shape such as “round” and “irregular form” can be mentioned. Also, for example, the relationship with the peripheral tissue, such as “pleural contact” and “pleural invagination”, and findings regarding the presence or absence of contrast, washout, and the like can be mentioned.

The position means an anatomical position, a position in a medical image, and a relative positional relationship with other regions of interest such as “inside”, “margin”, and “periphery”. The anatomical position may be indicated by an organ name such as “lung” and “liver”, and may be expressed in terms of subdivided lungs such as “right lung”, “upper lobe”, and apical segment (“S1”). The measurement value is a value that can be quantitatively measured from a medical image, and is, for example, at least one of a size or a signal value of a region of interest. The size is represented by, for example, a major axis, a minor axis, an area, a volume, or the like of a region of interest. The signal value is represented by, for example, a pixel value in a region of interest, a CT value in units of HU, or the like. The estimated disease name is an evaluation result estimated based on the lesion. Examples of the estimated disease name include a disease name such as “cancer” and “inflammation” and an evaluation result such as “negative/positive”, “benign/malignant”, and “mild/severe” related to disease names and properties.

In a situation where a plurality of creators (such as radiologists) share the task of creating an interpretation report, there may be cases in which each comment on findings includes synonyms. For example, one radiologist may use the word “spicula” while another radiologist may use a different word with the same meaning, such as “fluff-like”. In order to deal with such spelling variations due to synonyms, in a case in which each of two or more comments on findings registered in the report DB 8 includes synonyms, it is preferable that the registration unit 30 adds the same finding information to each of the two or more comments on findings (so-called normalization). For example, in FIG. 6, “spicula” included in the comment on findings No. 1 and “fluff-like” included in the comment on findings No. 2 are synonymous, and therefore the same finding information “spicula” is added to each comment on findings.

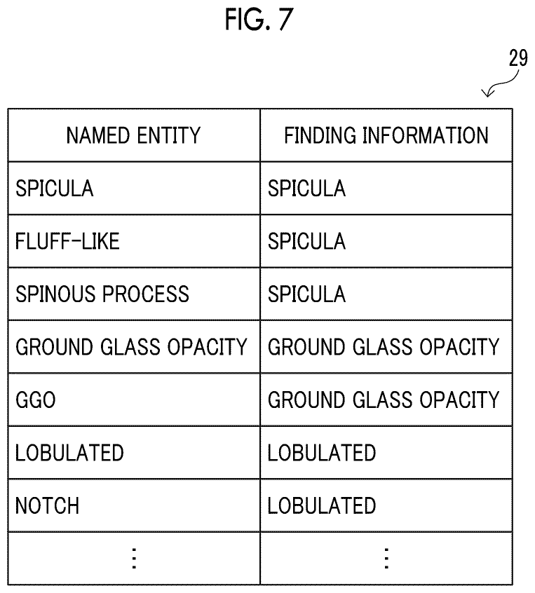

Specifically, a dictionary 29 in which a correspondence relationship between words indicating finding information that may be included in a comment on findings (so-called named entities) and the finding information is defined, in which synonymous named entities are associated with the same finding information, may be stored in advance in the storage unit 22. FIG. 7 shows an example of the dictionary 29. For example, in the dictionary 29, “spicula”, “fluff-like”, and “spinous process”, which indicate synonyms, are each associated with the same finding information, “spicula”. The registration unit 30 may extract named entities from the comments on findings registered in the report DB 8 and specify the finding information included in the comments on findings with reference to the dictionary 29. Note that, as a method for extracting named entities from a comment on findings, a known named entity extraction method using a natural language processing model such as, for example, bidirectional encoder representations from transformers (BERT) can be applied as appropriate.

Furthermore, the registration unit 30 may specify finding information based on a medical image related to the comment on findings, such as a key image and a medical image registered in the image DB 6 or the like. Specifically, the registration unit 30 may specify finding information that is not included in the comment on findings based on the medical image. The method of specifying the finding information based on the medical image is similar to a method of generating image finding information 62 by the generation unit 34, which will be described later.

For example, in a case in which the size of the region of interest is small, it may be less important to describe it or it may not be possible to measure it from the medical image, but in a case in which the size of the region of interest is large, it can be seen from the medical image. Therefore, the description may be omitted from the comment on findings regardless of its size. On the other hand, the size of the region of interest may be related to properties, etc., and even in a case in which the description is omitted from the comment on findings, it may implicitly affect the content of the comment on findings. For example, in lung cancer, the larger the tumor, the more likely it is that internal necrosis will occur. Therefore, it is preferable that the registration unit 30 adds finding information that is not included in the comment on findings but can be specified based on the medical image to the comment on findings and registers it in the report DB 8.

Furthermore, in a case in which one comment on findings registered in the report DB 8 includes finding information related to a plurality of regions of interest (lesions), it is preferable that the registration unit 30 specifies the finding information for each region of interest. For example, in FIG. 6, the comment on findings No. 3 includes a description of two nodules (regions of interest), and the finding information is also specified and registered for each nodule (region of interest).

In addition, it is preferable that the registration unit 30 also specifies the factuality of the specified finding information. The “factuality” is information indicating whether the finding is found or not, and the degree of certainty thereof and the like. This is because interpretation reports may include not only findings that are clearly found from medical images, but also findings that are not found from medical images, and findings that are suspicious but have a low degree of certainty. For example, the presence or absence of “calcification” may be used in the diagnosis of lung nodules, and the interpretation report may intentionally describe that “calcification is not observed” (see field No. 4 in FIG. 6).

In addition, the finding information may be information that modifies other finding information. In this case, it is preferable that the registration unit 30 also specifies a modification relationship between the pieces of finding information. For example, “calcification”, which is an example of the property of the lung nodule, may be described in detail as “microcalcification is observed in the center”. In this case, the registration unit 30 may specify the finding information of “center” and “micro” as other finding information that modifies the finding information of “calcification”. Examples of finding information that modifies “calcification” include “micro”, “coarse”, “scattered”, “center”, “ring-shaped”, and “complete”.

For example, in the case of “renal cell carcinoma”, which is an example of an estimated disease name based on the nodule of the kidney, a tissue type such as “clear cell type”, “papillary”, “chromophobe”, and “multilocular cystic” may also be described in the comments on findings. The registration unit 30 may specify the above-mentioned finding information indicating the tissue type as other finding information that modifies the finding information “renal cell carcinoma”.

Since the information processing apparatus 10 according to the present exemplary embodiment searches for a comment on findings using the finding information registered in the report DB 8, it is desirable to register as much finding information as possible in the report DB 8. The registration unit 30 specifies the finding information based on at least one of the comment on findings or the medical image, registers the finding information in the report DB 8, and makes it possible to use the finding information in searches, thereby contributing to improved search accuracy.

### (Supporting in Creation of Interpretation Report)

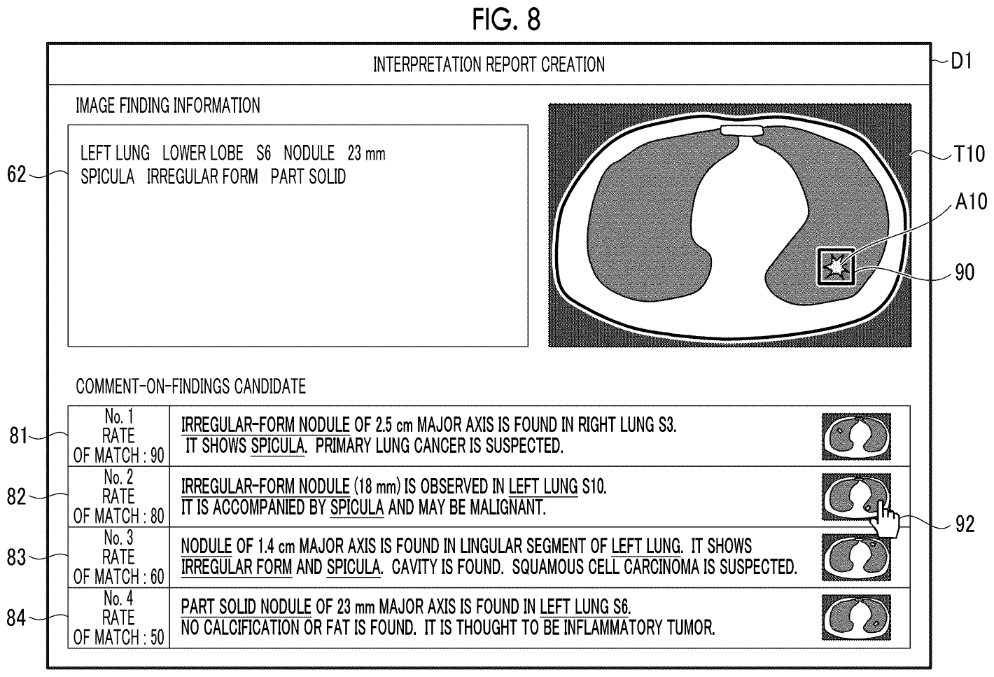

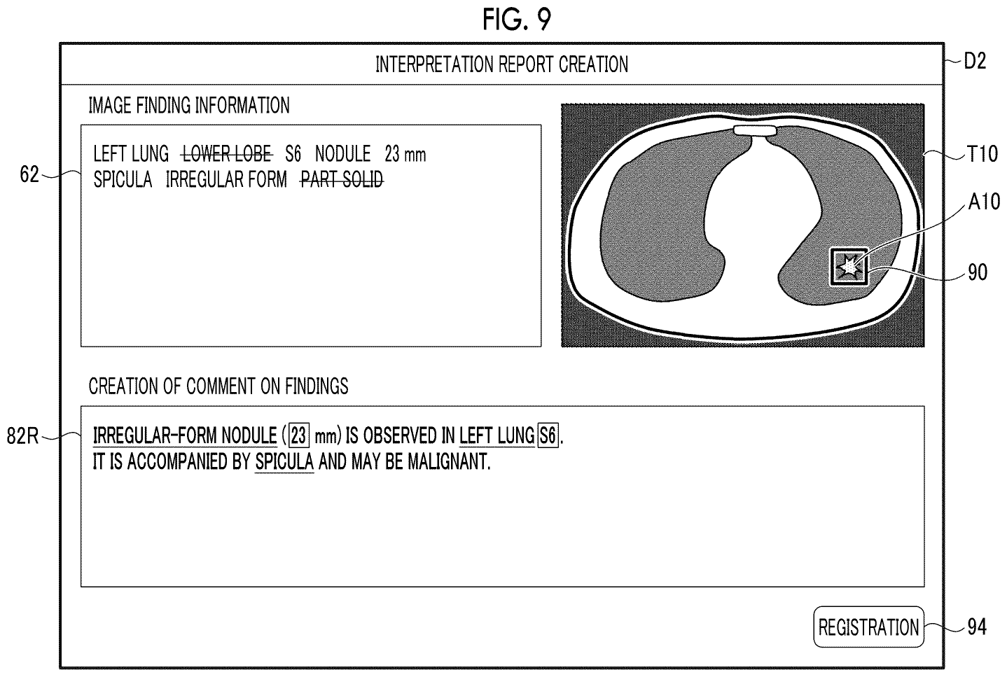

In this manner, the comment on findings, creator information, and key image registered when the interpretation report was created are registered in the report DB 8 in association with the finding information added by the registration unit 30. Using this information, the information processing apparatus 10 supports creating an interpretation report. Hereinafter, the function of the information processing apparatus 10 in supporting the creation of an interpretation report will be described with reference to FIGS. 8 and 9. FIG. 8 shows an example of a screen D1 displayed on the display 24 by the control unit 38 in the case of creating an interpretation report.

The acquisition unit 32 acquires a medical image T10 to be interpreted from the image server 5. The medical image acquired by the acquisition unit 32 may consist of one image, or include a plurality of images, such as the medical image T consisting of the tomographic images T1 to Tm in FIG. 2, for example.

The generation unit 34 generates, based on the medical image T10 acquired by the acquisition unit 32, at least one piece of image finding information 62 that is information indicating findings in the medical image T10. The image finding information 62 is, for example, information indicating at least one of a type (name), a property, a position, a measurement value, or an estimated disease name of a lesion included in the medical image T10. Details regarding the type (name), property, position, measurement value, and estimated disease name are similar to the finding information added to the comment on findings above, and therefore the description will be omitted.

Specifically, first, the generation unit 34 extracts at least one region of interest (lesion region A10 in FIG. 8) included in the medical image T10 acquired by the acquisition unit 32. As a method for extracting a region of interest, a method using known computer aided detection/diagnosis (CAD) technology and artificial intelligence (AI) technology and the like can be applied as appropriate. For example, the generation unit 34 may extract a region of interest from a medical image by using a learning model such as a convolutional neural network (CNN) that has been trained to receive the medical image as an input and extract and output a region of interest included in the medical image.

The generation unit 34 then generates, as image finding information 62, information indicating findings in the extracted region of interest (lesion region A10). For example, the generation unit 34 may generate image finding information of a region of interest by using a learning model such as a CNN that has been trained in advance to receive the region of interest extracted from the medical image as an input and output the image finding information of the region of interest. FIG. 8 shows an example in which a plurality of pieces of image finding information 62 such as “left lung” and “spicula” are generated as image finding information 62 for the lesion region A10.

In addition, in a case in which the subject of the medical image to be interpreted has been imaged and interpreted in the past for follow-up observation or the like, the generation unit 34 may generate information indicating changes in findings over time as the image finding information. Information indicating changes in findings over time is, for example, information indicating changes such as fluctuations in measurement values such as the size and signal values of a lesion, the appearance or disappearance of a lesion, and improvement or worsening of the property.

Specifically, the acquisition unit 32 acquires at least one of a past image obtained by capturing an image of the subject of the medical image to be interpreted in the past, or at least one piece of past finding information that is information indicating findings in the past image. The past images are registered in advance in, for example, the image DB 6. The past finding information is specified by the registration unit 30 based on, for example, an interpretation report created for a past image, and is registered in advance in the report DB 8.

The generation unit 34 generates image finding information indicating changes in findings over time based on the medical image of the interpretation target acquired by the acquisition unit 32 and at least one of the past image or the past finding information. For example, the generation unit 34 may generate image finding information indicating changes in findings over time by comparing the medical image T10 to be interpreted with a past image. For example, the generation unit 34 may extract a region of interest in a past image corresponding to the region of interest extracted from the medical image to be interpreted (hereinafter referred to as the “past region of interest”), and in a case in which the region of interest included in the medical image to be interpreted is larger than the past region of interest, generate image finding information indicating that its size tends to increase.

Furthermore, for example, the generation unit 34 may generate image finding information indicating changes in findings over time by comparing image finding information generated for the medical image to be interpreted with past finding information. For example, in a case in which image finding information indicating the size of a region of interest extracted from a medical image to be interpreted (for example, “23 mm”) is larger than past finding information indicating the size of a past region of interest (for example, “20 mm”), the generation unit 34 may generate image finding information indicating that the size tends to increase.

The search unit 36 searches the report DB 8 for at least one comment on findings including the image finding information 62 generated by the generation unit 34. Specifically, the search unit 36 searches the report DB 8 for a comment on findings to which finding information that is the same as or similar to at least one piece of image finding information 62 generated by the generation unit 34 has been added. FIG. 8 shows an example in which a plurality of comment-on-findings candidates 81 to 84, each of which includes at least one of the plurality of pieces of image finding information 62 generated by the generation unit 34, are output as search results. The comment-on-findings candidates 81 to 84 are examples of comments on findings including the image finding information of the present disclosure.

The search unit 36 may preferentially search for a comment on findings related to the image finding information 62 indicating a predetermined content among the plurality of pieces of image finding information 62 generated by the generation unit 34. For example, the search unit 36 may preferentially search for a comment on findings related to the image finding information 62 indicating the type, property, and estimated disease name of the lesion among the pieces of image finding information 62 indicating each of the type, property, position, measurement values, and estimated disease name of the lesion generated by the generation unit 34. On the other hand, the search unit 36 does not need to use the image finding information 62 indicating the position and the measurement value for the search. This is because positions and measurement values vary widely from subject to subject, and therefore using them in a search would result in fewer search results. On the other hand, the time and effort required for correcting the position and the measurement value in the comment on findings is minimal, and therefore it may be more efficient not to use them in a search.

Furthermore, in a case of searching for a comment on findings, the search unit 36 may allow ambiguity in the image finding information 62 used for the search. For example, for the image finding information 62 indicating a position, a comment on findings to which finding information indicating a position within a predetermined range (for example, the position of an anatomically adjacent area) is added may be output as a search result. Also, for example, for the image finding information 62 indicating a measurement value, a comment on findings to which finding information indicating a measurement value including a difference in a predetermined amount or percentage has been added may be output as a search result. For example, for the image finding information 62 indicating an estimated disease name, a comment on findings to which finding information indicating different estimated disease names belonging to the same classification (for example, “primary lung cancer” and “lung cancer”) is added may be output as a search result. In addition, the determination as to whether or not different estimated disease names belong to the same classification may be made using, for example, International Statistical Classification of Diseases and Related Health Problems (ICD) codes, dictionaries, ontologies, and the like.

In addition, in a case in which a comment on findings for a past image obtained by capturing an image of the subject of the medical image T10 to be interpreted in the past is registered in the report DB 8, the search unit 36 may preferentially search for the comment on findings. For example, the search unit 36 may refer to the image DB 6, and in a case in which a medical image in which a subject ID indicating the subject of the medical image T10 to be interpreted is attached is registered, the search unit 36 may search the report DB 8 for the interpretation report on the medical image. According to such a form, it becomes easier to standardize the expression method for each subject, and thus an easy-to-read interpretation report can be created even in a case of referring to a past interpretation report during follow-up observation, for example.

In addition, the search unit 36 may receive a user's designation of the creator of the comment on findings to be searched, and in a case in which a comment on findings to which creator information indicating the designated creator is added is registered in the report DB 8, the search unit 36 may preferentially search for that comment on findings. According to such a form, for example, by designating the user himself/herself, it is possible to search for comments on findings created by the user in the past, that is, comments on findings that the user likes. Also, by designating a representative creator, such as an experienced radiologist, it becomes easier to standardize the method of expressing comments on findings even in a case in which a plurality of radiologists share the interpretation work.

In addition, the search unit 36 may preferentially search for comments on findings described on medical images that have the same or similar information related to imaging (for example, imaging method, imaging conditions, imaging purpose, imaging location, imaging date and time, and the like) as the medical image T10 to be interpreted. Similarly, the search unit 36 may preferentially search for comments on findings described on medical images that have the same or similar information related to the subject (for example, age, gender, and medical history) as the medical image T10 to be interpreted. This information can be specified, for example, based on the accessory information attached to the medical image.

The search unit 36 may also calculate a rate of match between the image finding information 62 generated by the generation unit 34 and the finding information added to the searched-for comment-on-findings candidates 81 to 84. For example, the search unit 36 may calculate the percentage of finding information that is the same as or similar to the image finding information 62 included in the comments on findings obtained through the search as the rate of match. In this case, the search unit 36 may weigh the image finding information 62 according to its contents, for example by giving a greater weight to the image finding information 62 indicating the type, property, and estimated disease name of the lesion than to the image finding information 62 indicating the position and measurement values of the lesion.

As described above, finding information specified based on medical images, even in a case in which it is not included in the comments on findings may be added to the comments on findings registered in the report DB 8 (that is, the comment-on-findings candidates 81 to 84). In this case, the search unit 36 may calculate a rate of match only with the finding information included in the comment on findings, or may calculate a rate of match including finding information not included in the comment on findings that is specified based on the medical image. Furthermore, the search unit 36 may calculate both of these degrees of match.

The control unit 38 performs control to cause the display 24 to display the comment-on-findings candidates 81 to 84 obtained by the search unit 36 through the search. Moreover, it is preferable that the control unit 38 causes the display 24 to display the comment-on-findings candidates 81 to 84 obtained through the search and key images related to the comment-on-findings candidates 81 to 84 in association with each other. For example, as shown on the screen D1, the control unit 38 may display the comment-on-findings candidates 81 to 84 obtained by the search unit 36 through the search, and thumbnails of key images registered in the report DB 8 in association with each of the comment-on-findings candidates 81 to 84. Furthermore, for example, in a case in which a thumbnail of a key image is selected by the user operating a mouse pointer 92 via the input unit 25, the control unit 38 may enlarge and display the selected key image on the display 24.

Furthermore, in a case of displaying the medical image T10 to be interpreted, which is acquired by the acquisition unit 32, the control unit 38 may highlight the region of interest extracted by the generation unit 34. FIG. 8 shows an example in which a lesion region A10 is highlighted by a bounding box 90 in the medical image T10.

In addition, in a case in which the search unit 36 has calculated the rate of match between the image finding information 62 and the finding information added to the comment-on-findings candidates 81 to 84, the control unit 38 may determine a display method for the searched-for comment-on-findings candidates 81 to 84 based on the rate of match. In FIG. 8, the rate of match is displayed to the left of each of the comment-on-findings candidates 81 to 84. As shown in FIG. 8, the control unit 38 may display the comment-on-findings candidates 81 to 84 in a line in order of the rate of match. Additionally, for example, the control unit 38 may highlight the comments on findings whose rate of match is equal to or greater than a predetermined threshold value. Also, for example, the control unit 38 may display the number of pieces of finding information that are the same as or similar to the image finding information 62 and that have been added to the searched-for comment-on-findings candidates 81 to 84.

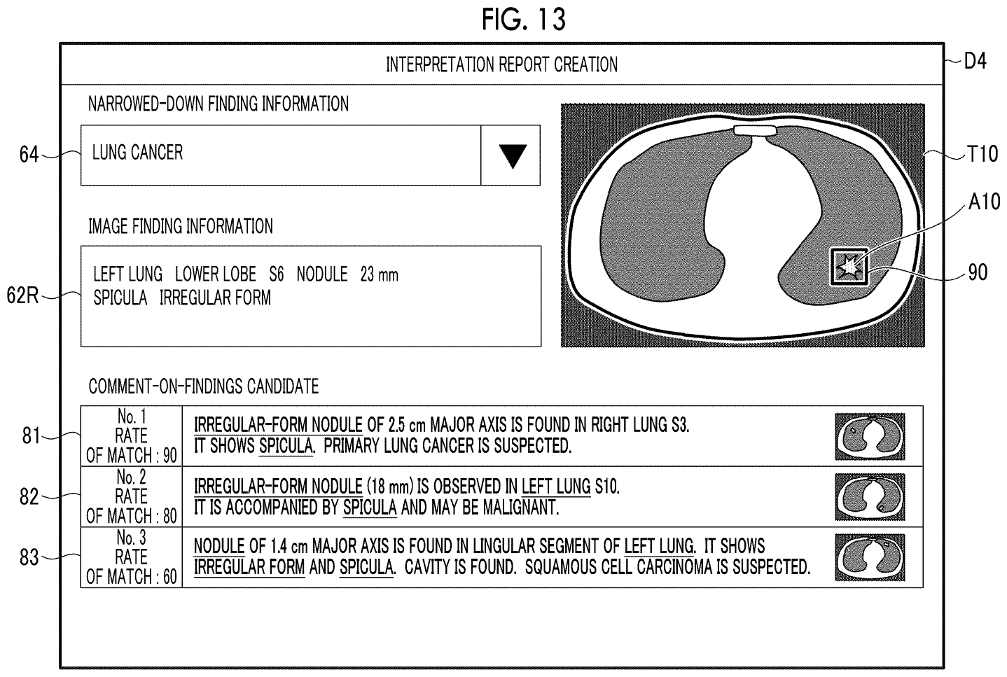

The control unit 38 may also perform control to cause the display 24 to display the portions of the comment-on-findings candidates 81 to 84 that correspond to the image finding information 62 in an identifiable manner. The expression “in an identifiable manner” means, for example, changing the thickness, size, italics, font, character color, and background color of the characters, attaching underlining, strikethrough, and enclosing lines, and displaying the character strings in different fields. In the example of FIG. 8, for each of the comment-on-findings candidates 81 to 84, the portion corresponding to the image finding information 62 is underlined. The same applies to FIGS. 9 and 13, which will be described later. According to such a form, it becomes easy to visually understand which image finding information 62 is included in each of the comment-on-findings candidates 81 to 84, and to what extent the finding information unrelated to the image finding information 62 is included. Therefore, in a case in which the user selects any one of the comment-on-findings candidates 81 to 84, the user can easily make a decision.

In addition, the control unit 38 may receive a selection of one of the comments on findings obtained by the search unit 36 through the search to be employed as the basis for the comments on findings for the medical image T10 to be interpreted, and may receive corrections to the selected comment on findings. For example, the user operates the mouse pointer 92 via the input unit 25 to select one of the comment-on-findings candidates 81 to 84 displayed on the screen D1 to be employed. FIG. 9 shows an example of a screen D2 displayed on the display 24 by the control unit 38 in a case in which the comment-on-findings candidate 82 of No. 2 is selected on the screen D1 in FIG. 8.

In addition, the control unit 38 may correct the comments on findings obtained through the search, based on the medical image T10 acquired by the acquisition unit 32. Specifically, the control unit 38 may use the image finding information 62 generated by the generation unit 34 based on the medical image T10 to correct the portion of the comment on findings obtained through the search that does not match the image finding information 62. In this case, the control unit 38 may also perform control to cause the display 24 to display the portion of the comments on findings that does not match the image finding information 62 in an identifiable manner. FIG. 9 shows, as an example, a comment on findings 82R in which the descriptions regarding the position and measurement values of one of the comment-on-findings candidates 82 have been corrected to contents corresponding to the image finding information 62. In the comment on findings 82R, enclosing lines are attached to the portions that do not match the image finding information 62 (descriptions regarding positions and measurement values).

The control unit 38 may also perform control to cause the display 24 to display the image finding information 62 that is not included in the comment on findings 82R out of the image finding information 62 generated by the generation unit 34 in an identifiable manner. In the example of FIG. 9, a strikethrough is attached to the image finding information 62 that is not included in the comment on findings 82R. In addition, the control unit 38 may update the presence or absence of a strikethrough in the image finding information 62 in a case in which the comment on findings 82R is corrected.

The user checks the comment on findings 82R displayed on the screen D2, corrects the comment on findings 82R via the input unit 25 as necessary, and then selects a registration button 94. In a case in which the registration button 94 is selected, the control unit 38 transmits, to the report server 7, a request to register the interpretation report including the corrected comment on findings 82R. In this case, the control unit 38 may request that the image finding information 62 generated by the generation unit 34 is also registered.

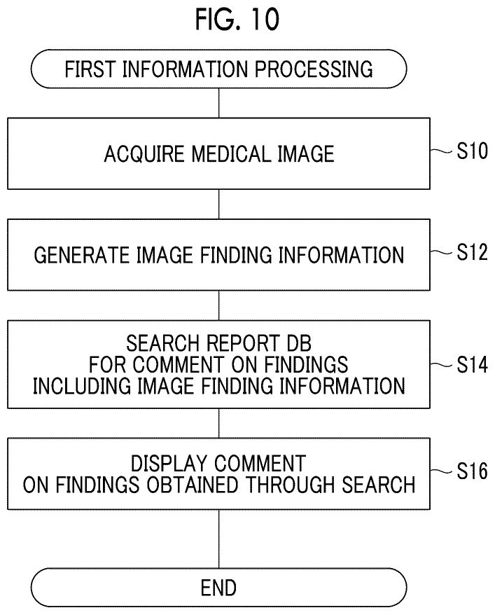

Next, with reference to FIG. 10, operations of the information processing apparatus 10 according to a first exemplary embodiment will be described. In the information processing apparatus 10, the CPU 21 executes the information processing program 27, and thus first information processing shown in FIG. 10 is executed. The first information processing is executed, for example, in a case in which the user provides an instruction to start execution via the input unit 25.

In Step S10, the acquisition unit 32 acquires a medical image to be interpreted from the image server 5. In step S12, the generation unit 34 generates at least one piece of image finding information, which is information indicating findings in the medical image, based on the medical image acquired in step S10. In step S14, the search unit 36 searches the report DB 8 for at least one comment on findings to which the image finding information generated in step S12 has been added. In Step S16, the control unit 38 performs control to display, on the display 24, the comment on findings obtained through the search in Step S14, and ends this information processing.

As described above, the information processing apparatus 10 according to one aspect of the present disclosure comprises at least one processor, and the processor acquires an image, generates at least one piece of image finding information, which is information indicating findings in the image, based on the image, and searches for at least one comment on findings related to the image finding information from a database in which a plurality of comments on findings are registered in advance.

That is, the information processing apparatus 10 according to the first exemplary embodiment can search for a comment on findings that corresponds to the medical image to be interpreted and that has been registered in the report DB 8. Therefore, since the method of expressing comments on findings can be reused as it is, the creation of an interpretation report can be supported without causing the user the time and effort of correcting the expression of the sentences.

In the above first exemplary embodiment, an example in which the medical image T10 includes one lesion region A10 has been described, but the present disclosure is not limited thereto. For example, a medical image may include a plurality of lesion regions. In a case in which a plurality of regions of interest included in a medical image are extracted, it is preferable that the generation unit 34 generates image finding information for each region of interest. That is, it is preferable that the generation unit 34 generates image finding information indicating at least one of the type, property, position, measurement value, or estimated disease name for each region of interest, as shown in No. 3 of FIG. 8.

In this case, it is preferable that the search unit 36 searches the report DB 8 for comments on findings related to a plurality of pieces of image finding information for each region of interest. That is, it is preferable that the search unit 36 searches for a comment on findings that includes descriptions regarding a plurality of regions of interest, as shown in No. 3 in FIG. 8, and is added with finding information that is as same or similar as possible to a plurality of pieces of image finding information for each region of interest.

In the above first exemplary embodiment, as shown in FIGS. 8 to 9, a form has been described in which the control unit 38 corrects the selected comment-on-findings candidate 82 from among the comment-on-findings candidates 81 to 84 obtained by the search unit 36 through the search, but the present disclosure is not limited thereto. For example, on the screen D1, the control unit 38 may perform control to display the comment-on-findings candidates 81 to 84 on the display 24 after correcting each of the comment-on-findings candidates 81 to 84. Also, for example, the control unit 38 may switchably display, on the display 24, either the comment on findings obtained through the search (that is, the original sentence) or the corrected comment on findings.

In addition, in the above first exemplary embodiment, a form has been described in which the generation unit 34 extracts at least one region of interest included in a medical image and generates image finding information indicating findings of the extracted region of interest, but the present disclosure is not limited thereto. For example, the generation unit 34 may receive a designation of at least one region of interest included in a medical image. Specifically, the generation unit 34 may display the medical image to be interpreted on the display 24 and receive a user's designation of the position and range of a region of interest in the medical image via the input unit 25. In this case, the generation unit 34 may generate information indicating findings in the designated region of interest as image finding information.

In this case, the region of interest receiving a designation is not limited to one, and a designation of a plurality of regions of interest included in the medical image may be received. In this case, it is preferable that the generation unit 34 generates image finding information for each region of interest. In this case, it is preferable that the search unit 36 searches the report DB 8 for comments on findings related to a plurality of pieces of image finding information for each region of interest.

### Second Exemplary Embodiment

In the above first exemplary embodiment, a form has been described in which a search is performed using image finding information generated by the generation unit 34 based on a medical image. In this case, as shown in FIG. 8, various variations of comments on findings, such as comment-on-findings candidates 81 and 82 having the same content but different methods of expression, and comment-on-findings candidates 83 and 84 having different content, may be output as search results. For example, in a case in which a radiologist is unsure how to interpret a medical image, it is preferable to present many variations of comments on findings, which differ not only in the methods of expression but also in the content, as shown in FIG. 8.

On the other hand, for example, in a case in which a radiologist is able to interpret a medical image immediately, it is desirable to present only a comment on findings that is narrowed down in accordance with the contents of the interpretation by the radiologist. Therefore, an information processing apparatus 10 according to a second exemplary embodiment has a function of narrowing down image finding information used in a search, in addition to the function described in the first exemplary embodiment. Hereinafter, functions of the information processing apparatus 10 according to the second exemplary embodiment will be described with reference to the drawings, but some of the descriptions overlapping with those of the first exemplary embodiment will be omitted.

As in the first exemplary embodiment, the registration unit 30 adds, to each of the plurality of comments on findings registered in the report DB 8, finding information related to the comment on findings (see FIG. 6). The acquisition unit 32 acquires a medical image T10 to be interpreted from the image server 5.

The control unit 38 performs control to display the medical image T10 acquired by the acquisition unit 32 on the display 24. Then, the control unit 38 receives an input of at least one piece of narrowed-down finding information regarding the medical image T10 to be interpreted that has been acquired by the acquisition unit 32. In this case, the control unit 38 may present candidates for the narrowed-down finding information and receive the selection of at least one of them. The narrowed-down finding information is, for example, information indicating the estimated disease name of the lesion included in the medical image T10.

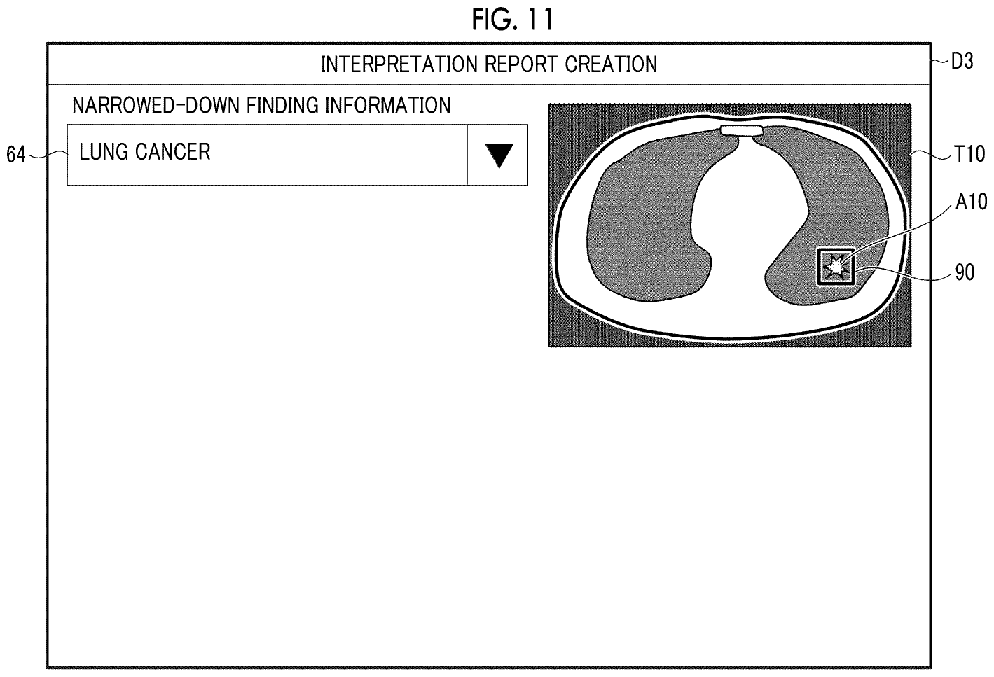

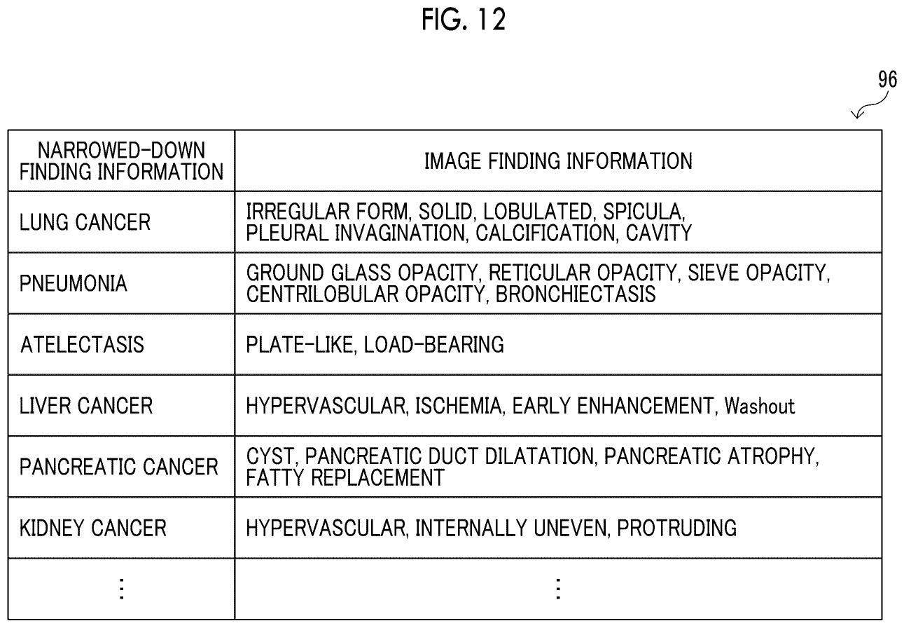

FIG. 11 shows an example of a screen D3 for receiving an input of the narrowed-down finding information 64, which is displayed on the display 24 by the control unit 38. In FIG. 11, “lung cancer” is input as the narrowed-down finding information 64. As shown in FIG. 11, the control unit 38 may receive an input of narrowed-down finding information, for example, via a pull-down menu that enables a designation of various predetermined estimated disease names (see FIG. 12). The user interprets the medical image T10 displayed on the display 24 and inputs the narrowed-down finding information 64 via the input unit 25.

The generation unit 34 generates information indicating findings in the medical image T10 related to the narrowed-down finding information 64 as image finding information 62R of the medical image T10 to be interpreted. FIG. 12 shows an example of a table 96 in which the narrowed-down finding information is associated with the image finding information related to each piece of narrowed-down finding information. The table 96 is stored in advance in the storage unit 22, for example.

Specifically, the generation unit 34 generates image finding information 62R that can be generated based on the medical image T10 to be interpreted and that is determined in the table 96 to be related to the narrowed-down finding information 64. For example, as shown in FIG. 8, based on the medical image T10, image finding information 62 indicating the properties of the lesion region A10 can be generated as three types: “spicula”, “irregular form” and “part solid”. On the other hand, in the table 96 of FIG. 12, the image finding information corresponding to “lung cancer” includes “spicula” and “irregular form”, but does not include “part solid”. In this case, the generation unit 34 generates information indicating “spicula” and “irregular form” as the image finding information 62R of the medical image T10, but does not generate information indicating “part solid”.

The search unit 36 searches the report DB 8 for a comment on findings to which at least one of the image finding information 62R or the narrowed-down finding information 64 has been added. FIG. 13 shows an example of a screen D4 on which comment-on-findings candidates 81 to 83 including at least one of the image finding information 62R indicating “spicula” and “irregular form” or the narrowed-down finding information 64 indicating “lung cancer” are output as search results. The screen D4 does not include the comment-on-findings candidate 84 in FIG. 8. This is because the comment-on-findings candidate 84 did not include the image finding information 62R indicating “spicula” and “irregular form”, but only included the finding information indicating “part solid” that was excluded based on the narrowed-down finding information 64.

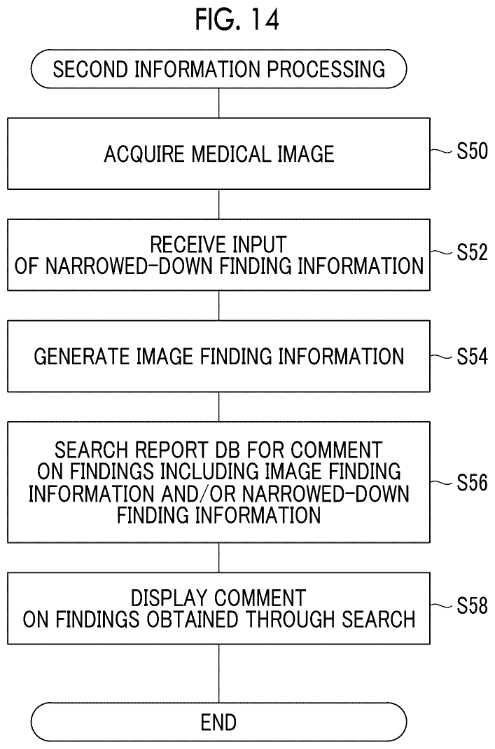

Next, with reference to FIG. 14, operations of the information processing apparatus 10 according to a second exemplary embodiment will be described. In the information processing apparatus 10, the CPU 21 executes the information processing program 27, and thus second information processing shown in FIG. 14 is executed. The second information processing is executed, for example, in a case in which the user provides an instruction to start execution via the input unit 25.

In Step S50, the acquisition unit 32 acquires a medical image to be interpreted from the image server 5. In step S52, the control unit 38 performs control to display the medical image acquired in step S50 on the display 24, and receives an input of at least one piece of narrowed-down finding information related to the medical image. In step S54, the generation unit 34 generates information indicating findings in the medical image related to the narrowed-down finding information received in step S52, as image finding information of the medical image acquired in step S50.

In step S56, the search unit 36 searches the report DB 8 for a comment on findings to which at least one of the image finding information generated in step S54 or the narrowed-down finding information received in step S52 has been added. In Step S58, the control unit 38 performs control to display, on the display 24, the comment on findings obtained through the search in Step S56, and ends this information processing.

As described above, the information processing apparatus 10 according to one aspect of the present disclosure comprises at least one processor, and the processor acquires an image, generates at least one piece of image finding information, which is information indicating findings in the image, based on the image, and searches for at least one comment on findings related to the image finding information from a database in which a plurality of comments on findings are registered in advance. The processor also receives an input of at least one piece of narrowed-down finding information related to the image, and generates, as the image finding information, information indicating findings of the image related to the narrowed-down finding information.

That is, with the information processing apparatus 10 according to the second exemplary embodiment, it is possible to search for comments on findings that correspond to both the medical image to be interpreted (image finding information) and the content of the interpretation by the radiologist (narrowed-down finding information) and that have been registered in the report DB 8. Therefore, since the method of expressing comments on findings can be reused as it is while further improving the search accuracy, the creation of an interpretation report can be supported without causing the user the time and effort of correcting the expression of the sentences.

In the above second exemplary embodiment, a form has been described in which the narrowed-down finding information 64 is input using a pull-down menu, but the present disclosure is not limited thereto. For example, the control unit 38 may receive an input of a partial comment on findings that is a part of a comment on findings related to a medical image and that includes narrowed-down finding information, and specify the narrowed-down finding information included in the partial comment on findings. For example, in a case in which an input of a partial comment on findings such as “lung cancer is suspected” is received, the control unit 38 may perform a named entity extraction process and specify “lung cancer”, which is one of the narrowed-down finding information items predetermined in the table 96 (see FIG. 12). As a method for extracting named entities from partial comments on findings, a known named entity extraction method using a natural language processing model such as BERT can be appropriately applied.

In the above second exemplary embodiment, a form has been described in which the narrowed-down finding information is the finding information indicating an estimated disease name (see FIG. 12), but the present disclosure is not limited thereto. As the narrowed-down finding information, for example, finding information indicating at least one of a type (name), a property, a position, a measurement value, or an estimated disease name of the lesion included in the medical image T10 can be appropriately applied.

For example, the narrowed-down finding information in FIG. 12 includes estimated disease names (for example, “lung cancer” and “liver cancer”) that differ in anatomical position (for example, “lung” and “liver”). Instead of this, for example, information indicating an estimated disease name determined in advance for each anatomical position of a lesion may be used as the narrowed-down finding information. Specifically, the control unit 38 may specify the anatomical position of the lesion based on the image finding information generated by the generation unit 34. In addition, the control unit 38 may receive an input of narrowed-down finding information via a pull-down menu that enables a designation of an estimated disease name according to the specified anatomical position (for example, in the case of the lungs, “lung cancer”, “pneumonia”, and “atelectasis”).

In the second exemplary embodiment, the control unit 38 may receive inputs of a plurality of pieces of narrowed-down finding information. In this case, the search unit 36 searches the report DB 8 for image finding information and a comment on findings to which at least one of the plurality of pieces of narrowed-down finding information has been added. The means for receiving inputs of a plurality of pieces of narrowed-down finding information is not particularly limited. For example, this may be realized by displaying, on the display 24, a graphical user interface (GUI) that enables a designation of a plurality of items, such as a multi-select list and check boxes. Also, for example, this may be realized by displaying, on the display 24, a GUI, such as a free-form text box, into which the user can input a plurality of pieces of narrowed-down finding information or partial comments on findings including a plurality of pieces of narrowed-down finding information. Also, for example, this may be realized by displaying, on the display 24, a GUI such as a slider that enables a designation of the size of the region of interest and the range of measurement values such as signal values (that is, a maximum value and a minimum value).

Furthermore, the control unit 38 may receive inputs of a plurality of pieces of narrowed-down finding information in stages. Specifically, the control unit 38 may receive an input of first narrowed-down finding information, and then receive an input of second narrowed-down finding information related to the first narrowed-down finding information. In this case, the control unit 38 may change the content presented as candidates for the second narrowed-down finding information depending on the first narrowed-down finding information. For example, in a case in which “renal cell carcinoma” is input as the first narrowed-down finding information, the control unit 38 may receive an input of at least one of “clear cell type”, “papillary”, “chromophobe”, or “multilocular cystic”, as the second narrowed-down finding information indicating the tissue type of “renal cell carcinoma”.

In the above second exemplary embodiment, a form has been described in which the candidate for the narrowed-down finding information is predetermined in the table 96, but the present disclosure is not limited thereto. For example, the control unit 38 may search the report DB 8 for comments on findings to which the image finding information generated by the generation unit 34 has been added, count the finding information added to the comments on findings obtained as the search results, and select the information as candidates for narrowed-down finding information in descending order of frequency. In addition, for example, after receiving the input of the first narrowed-down finding information, the control unit 38 may search the report DB 8 for the comment on findings to which the first narrowed-down finding information has been added, count the finding information added to the comment on findings obtained as the search result, and select the information as candidates for the second narrowed-down finding information in descending order of frequency. In these cases, the types of finding information to be counted (that is, finding information that can be candidates for narrowed-down finding information) may be limited. For example, only the finding information indicating the type (name) of the lesion and the estimated disease name may be counted, and the finding information indicating the property, position, and measurement value may be excluded from the counting.

Furthermore, in the above second exemplary embodiment, in a case of presenting candidates for the narrowed-down finding information, the control unit 38 may present information that assists the user in selecting the narrowed-down finding information. For example, the control unit 38 may present the number of comment-on-findings candidates output as a search result in a case of searching for comments on findings using the narrowed-down finding information. Furthermore, for example, the control unit 38 may present the percentage of comments on findings to which narrowed-down finding information has been added, among the comments on findings registered in the report DB 8. Further, for example, in a case in which the input of the first narrowed-down finding information and the second narrowed-down finding information is received in stages, the control unit 38 may present at least one of the number or the percentage of the comment-on-findings candidates to which the second narrowed-down finding information has been added, among the comment-on-findings candidates to which the first narrowed-down finding information has been added.

In the above second exemplary embodiment, a form has been described in which the generation unit 34 generates the image finding information 62R related to the narrowed-down finding information 64, the search unit 36 searches for the comment on findings based on the image finding information 62R, and the control unit 38 displays the image finding information 62R on the display 24 (see FIG. 13), but the present disclosure is not limited thereto. For example, the generation unit 34 may generate both all image finding information 62 (see FIG. 8) that can be generated based on a medical image T10 to be interpreted similar to the first exemplary embodiment, and image finding information 62R related to narrowed-down finding information 64 out of the image finding information 62 (see FIG. 8). In addition, the control unit 38 may perform control to cause the display 24 to display the image finding information 62R related to the narrowed-down finding information 64 in an identifiable manner, out of all the image finding information 62 that can be generated based on the medical image T10 to be interpreted. The expression “in an identifiable manner” means, for example, changing the thickness, size, italics, font, character color, and background color of the characters, attaching underlining, strikethrough, and enclosing lines, and displaying the character strings in different fields.

The control unit 38 may also perform control to display, on the display 24, a list of image finding information that is determined in the table 96 (see FIG. 12) in relation to the narrowed-down finding information 64 for which the input is received. For example, in a case in which “lung cancer” is input as the narrowed-down finding information 64, the control unit 38 may perform control to display, on the display 24, “irregular form, solid, lobulated, spicula, pleural invagination, calcification, and cavity” defined in the table 96 as image finding information corresponding to “lung cancer”. The control unit 38 may also perform control to cause the display 24 to display the image finding information 62R generated by the generation unit 34 in an identifiable manner from this list.

In each of the exemplary embodiments described above, the user may be able to narrow down the image finding information and the narrowed-down finding information used in the search. For example, in the screen D1 of FIG. 8 and the screen D3 of FIG. 13, at least one of the image finding information 62 or the image finding information 62R may be selectable, and the search unit 36 may re-search for the comment on findings based on the selected image finding information 62 or 62R. In addition, in a case in which the control unit 38 transmits a request to register an interpretation report to the report server 7, it may request that the image finding information 62 or 62R selected by the user is registered (that is, that the image finding information 62 or 62R that was not selected is deleted and registered).

In addition, in each of the exemplary embodiments described above, the user may be able to designate image finding information and narrowed-down finding information to be excluded from the search results. That is, the search unit 36 may not output, as a search result, image finding information designated to be excluded and comments on findings to which narrowed-down finding information has been added. For example, in a case in which it is designated to exclude image finding information indicating “spicula”, the search unit 36 may not output a comment on findings to which finding information indicating “spicula” has been added as a search result, regardless of other finding information and the rate of match, and the like.

Further, in each of the above exemplary embodiments, a form assuming interpretation for medical images has been described, but the present disclosure is not limited thereto. The information processing apparatus 10 according to one aspect of the present disclosure can be applied to various images including a region of interest, which are obtained by imaging a subject. For example, the information processing apparatus 10 may be applied to an image acquired using an apparatus, a building, a pipe, a welded portion, or the like as a subject in a non-destructive examination such as a radiation transmission examination and an ultrasonic flaw detection examination. In this case, for example, the region of interest indicates cracks, flaws, bubbles, foreign matter, or the like.

In addition, in each of the above exemplary embodiments, for example, as hardware structures of processing units that execute various kinds of processing, such as the registration unit 30, the acquisition unit 32, the generation unit 34, the search unit 36, and the control unit 38, various processors shown below can be used. As described above, the various processors include a programmable logic device (PLD) as a processor of which the circuit configuration can be changed after manufacture, such as a field programmable gate array (FPGA), a dedicated electrical circuit as a processor having a dedicated circuit configuration for executing specific processing such as an application specific integrated circuit (ASIC), and the like, in addition to the CPU as a general-purpose processor that functions as various processing units by executing software (programs).

One processing unit may be configured by one of the various processors, or may be configured by a combination of the same or different kinds of two or more processors (for example, a combination of a plurality of FPGAs or a combination of the CPU and the FPGA). In addition, a plurality of processing units may be configured by one processor.

As an example in which a plurality of processing units are configured by one processor, first, there is a form in which one processor is configured by a combination of one or more CPUs and software as typified by a computer, such as a client or a server, and this processor functions as a plurality of processing units. Second, as represented by a system on chip (SoC) or the like, there is a form of using a processor for realizing the function of the entire system including a plurality of processing units with one integrated circuit (IC) chip. In this way, various processing units are configured by one or more of the above-described various processors as hardware structures.

Furthermore, as the hardware structure of the various processors, more specifically, an electrical circuit (circuitry) in which circuit elements such as semiconductor elements are combined can be used.

In the above exemplary embodiment, the information processing program 27 is described as being stored (installed) in the storage unit 22 in advance; however, the present disclosure is not limited thereto. The information processing program 27 may be provided in a form recorded in a recording medium such as a compact disc read only memory (CD-ROM), a digital versatile disc read only memory (DVD-ROM), and a Universal Serial Bus (USB) memory. In addition, the information processing program 27 may be configured to be downloaded from an external device via a network. Further, the technology of the present disclosure extends to a storage medium for storing the information processing program non-transitorily in addition to the information processing program.

The technology of the present disclosure can be appropriately combined with the above exemplary embodiment and examples. The described contents and illustrated contents shown above are detailed descriptions of the parts related to the technology of the present disclosure, and are merely an example of the technology of the present disclosure. For example, the above description of the configuration, function, operation, and effect is an example of the configuration, function, operation, and effect of the parts related to the technology of the present disclosure. Therefore, it goes without saying that unnecessary parts may be deleted, new elements may be added, or replacements may be made to the described contents and illustrated contents shown above within a range that does not deviate from the gist of the technology of the present disclosure.

The disclosures of JP2022-154170 filed on Sep. 27, 2022 and JP2023-159178 filed on Sep. 22, 2023 are incorporated herein by reference in their entirety. All documents, patent applications, and technical standards described in the present specification are incorporated in the present specification by reference to the same extent as in a case in which each of the documents, patent applications, and technical standards are specifically and individually indicated to be incorporated by reference.

What is claimed is:

1. An information processing apparatus comprising a processor, wherein the processor is configured to:
acquire an image;
generate at least one piece of image finding information, which is information indicating findings in the image, based on the image; and
search for at least one comment on findings related to the image finding information from a database in which a plurality of comments on findings are registered in advance.

2. The information processing apparatus according to claim 1, wherein the processor is configured to:
receive an input of at least one piece of narrowed-down finding information related to the image; and
generate, as the image finding information, information indicating findings of the image related to the narrowed-down finding information.

3. The information processing apparatus according to claim 2, wherein the processor is configured to search the database for a comment on findings related to at least one of the image finding information or the narrowed-down finding information.

4. The information processing apparatus according to claim 2, wherein the processor is configured to:
receive an input of a partial comment on findings which is a part of a comment on findings related to the image and includes the narrowed-down finding information; and
specify the narrowed-down finding information included in the partial comment on findings.

5. The information processing apparatus according to claim 1, wherein the processor is configured to correct the comment on findings obtained through the search based on the image.

6. The information processing apparatus according to claim 1, wherein the processor is configured to:
acquire at least one of a past image obtained by capturing a subject of the image in the past or at least one piece of past finding information which is information indicating findings in the past image; and
generate the image finding information indicating changes in findings over time based on the image and at least one of the past image or the past finding information.

7. The information processing apparatus according to claim 1, wherein the processor is configured to:
extract at least one region of interest included in the image; and
generate information indicating findings of the extracted region of interest as the image finding information.

8. The information processing apparatus according to claim 7, wherein the processor is configured to, in a case in which a plurality of regions of interest included in the image are extracted:
generate the image finding information for each region of interest; and
search the database for comments on findings related to a plurality of pieces of the image finding information for each region of interest.

9. The information processing apparatus according to claim 1, wherein the processor is configured to:
receive a designation of at least one region of interest included in the image; and
generate information indicating findings of the designated region of interest as the image finding information.

10. The information processing apparatus according to claim 9, wherein the processor is configured to, in a case in which a designation of a plurality of regions of interest included in the image is received:
generate the image finding information for each region of interest; and
search the database for comments on findings related to a plurality of pieces of the image finding information for each region of interest.

11. The information processing apparatus according to claim 1, wherein the processor is configured to:
specify, for each of the plurality of comments on findings registered in the database, finding information related to a comment on findings and add the finding information to the comment on findings; and
search the database for a comment on findings to which finding information that is the same as or similar to the image finding information has been added.

12. The information processing apparatus according to claim 11, wherein the processor is configured to, in a case in which each of two or more comments on findings registered in the database includes a synonym, add the same finding information to each of the two or more comments on findings.

13. The information processing apparatus according to claim 1, wherein:
the plurality of comments on findings and key images related to the comments on findings are registered in advance in the database in association with each other, and
the processor is configured to display, on a display, the comment on findings obtained through the search and the key image related to the comment on findings in association with each other.

14. The information processing apparatus according to claim 1, wherein the processor is configured to:
calculate a rate of match between the image finding information and finding information included in the searched-for comment on findings; and
determine a display method for the searched-for comment on findings based on the rate of match.

15. The information processing apparatus according to claim 1, wherein the processor is configured to, in a case in which a comment on findings for a past image obtained by capturing a subject of the image in the past is registered in the database, preferentially search for the comment on findings.

16. The information processing apparatus according to claim 1, wherein:
creator information indicating a creator of a comment on findings is added to the plurality of comments on findings registered in the database, and
the processor is configured to:
receive a designation of a creator of a comment on findings to be searched; and
in a case in which a comment on findings to which the creator information indicating the designated creator is added is registered in the database, preferentially search for the comment on findings.

17. The information processing apparatus according to claim 1, wherein:
the image is a medical image, and
the image finding information indicates at least one of a type, a property, a position, a measurement value, or an estimated disease name of a lesion included in the medical image.

18. The information processing apparatus according to claim 2, wherein:
the image is a medical image, and
the narrowed-down finding information indicates an estimated disease name of a lesion included in the medical image.

19. An information processing method executed by a computer, the information processing method comprising:
acquiring an image;
generating at least one piece of image finding information, which is information indicating findings in the image, based on the image; and
searching for at least one comment on findings related to the image finding information from a database in which a plurality of comments on findings are registered in advance.

20. A non-transitory computer-readable storage medium storing an information processing program for causing a computer to execute:
acquiring an image;
generating at least one piece of image finding information, which is information indicating findings in the image, based on the image; and
searching for at least one comment on findings related to the image finding information from a database in which a plurality of comments on findings are registered in advance.
